(19) United States
(12) Patent Application Publication
(10) Pub. No.: US 2019/0048392 A1
(43) Pub. Date: Feb. 14, 2019

(54) APPARATUS AND METHOD FOR CONVERTING ELECTROMAGNETIC RADIATION INTO THERMAL ENERGY

(71) Applicant: IfP Privates Institut für Produktqualität GmbH, Berlin (DE)

(72) Inventors: Wolfgang Weber, Berlin (DE); Marcel Mallah, Peine (DE)

(73) Assignee: IfP Privates Institut für Produktqualität GmbH, Berlin (DE)

(21) Appl. No.: 15/567,691

(22) PCT Filed: Apr. 25, 2016
(86) PCT No.: PCT/EP2016/059176

§ 371 Date: Oct. 19, 2017

(30) Foreign Application Priority Data
Apr. 24, 2015 (DE) 10 2015 106 346.8

(51) Int. Cl. C12Q 1/6806; H05B 6/80; H05B 6/64; C12N 15/10

(52) U.S. Cl. CPC C12Q 1/6806; C12N 15/1003; H05B 6/647; H05B 6/806

(57) ABSTRACT

Apparatus for converting electromagnetic radiation into thermal energy for use in isolating nucleic acids for subsequent analysis by a DNA polymerase chain reaction, comprising an electromagnetic radiation generator, which emits microwaves; a chamber, which is connected to the generator and confines the emitted microwaves; a plurality of stationary spots, which are fixedly attached onto the upper part of the chamber and project inwards, wherein the stationary spots are longitudinally separated from each other by identical intervals of predetermined distance, and the emitted electromagnetic radiation propagates within the chamber as a standing wave, and wherein the said predetermined distance is half the wavelength of the emitted radiation.

This application is the national phase under 35 U.S.C. § 371 of PCT International Application No. PCT/EP2016/059176, which has an International filing date of Apr. 25, 2016, which designated the United States of America.

### FIELD OF THE INVENTION

The invention relates to an apparatus for converting electromagnetic radiation into thermal energy and a method of isolating nucleic adds for subsequent analysis by a DNA polymerase chain reaction encompassing the conversion of electromagnetic radiation into thermal energy.

### BACKGROUND OF THE INVENTION

Isolation of genomic DNA can be performed by the conventional method involving the use of the cationic surfactant hexadecyltrimethylammonium bromide (CTAB) for denaturing and removal of proteins (Drabkova L Z et al., DNA extraction from herbarium specimens, Methods Mol. Biol. 30 2014; 1115:69-84). This method requires the use of an expensive and hazardous chemical and is also laborious and time consuming. On the other hand, dissociation of samples for isolation of cellular components, such as nucleic acids for subsequent amplification and analysis by PCR, can be enhanced by application of heat. The treatment of a sample with heat and the addition of chemicals for isolating nucleic acids are described in US 2014/0051088. The described compounds represent, already at low concentration, a source of polymerase chain reaction inhibitors, which interferes with the enzymatic reaction.

The transfer of thermal energy occurs rather slowly by means of heating blocks or water baths. Fast heating of the sample is preferred for an accelerated dissociation process. EP 1 728 074 B1 discloses a process for extraction of intercellular complexes by drying large liquid samples and, subsequently, exposing the dried sample to microwaves. However, EP 1 728 074 B1 fails to disclose means for control of temperature within liquid samples upon exposure to microwaves. US 2009/0186 357 A1 (Mauk et al) claims a heating of the liquid sample by microwaves but fails to disclose any specific device or implementation. WO 01/19963 A2 (Motorola) describes a computer-controlled heating of small biological volumina by waves of 18 to 26 GHz, as well as specific wave guiding channels. The corresponding device is very complex and intricate. WO2010/141921 discloses a PCR reaction channel with a microwave module. Marchiarullo D J et al in Lab Chip (2013) 13:3417-3425 und Miralles V et al in Diagnostics (2013) 3:33-67 describe chips und mikrofluid systems and contemplete heating by microwaves. The increase in temperature of an aqueous solution by microwave radiation is linked to the high risk of bursting due to a sudden increase in vapor pressure or phase transition, with especial consideration of a liquid confined in a closed vessel or a microfluid system

US 2006/0141556 A1 describes a method of cell disruption by treating a sample with microwaves and addition of expensive zwitterionic surfactants. However, the disclosure fails to show a fast method by which cell lysis of difficult-to-dissociate samples, for example foodstuffs, can effectively be carried out inside closed vessels without bursting. Thus, the described method cannot prevent cross-contamination due to evaporation and spillage. EP 1 355 735 B1 discloses a system for microwave-assisted chemical synthesis comprising a round cavity and a microwave-attenuator, whereby only one single sample can be processed at a time. DE 100 16 962 C2 discloses an apparatus for dissociation of chemicals by means of microwaves. Yet this apparatus requires operation under pressure and displays rotatable containers for homogenous temperature distribution within the sample to be dissociated, thus increasing the complexity of the apparatus and performance of the dissociation process. The prior art therefore represents a problem.

### SUMMARY OF THE INVENTION

The problem is solved by the apparatus and method according to claims 1 and 11. Preferred embodiments of the invention are disclosed in the dependent claims.

The apparatus for converting electromagnetic radiation into thermal energy, comprises an electromagnetic radiation generator, which emits microwaves; a chamber, which is connected to the microwave generator and confines the emitted microwaves; a plurality of stationary spots, which are fixedly attached onto the upper part of the chamber and project inwards, wherein the stationary spots are longitudinally separated from each other by identical intervals of predetermined distance, and the emitted electromagnetic radiation propagates within the chamber as a standing wave. Said intervals correspond to the wavelength of the emitted radiation.

In a preferred embodiment, the apparatus can generate microwaves adopting a monomode pattern. The location of said spots may correspond to the maxima of the generated standing microwave pattern. In another embodiment, the wavelength of the radiation can range from about 3 to about 15 cm, preferably from about 4 cm to 13 cm, most preferably from about 5 cm to 11 cm.

In a further embodiment, the apparatus can exhibit spots comprising each an outer wall made of microwave-reflecting material and an inner holding piece made of a microwave transparent material such as plastic, silicon carbide, resin, Teflon®, polytetrafluoroethylene, perfluoroalkoxy, fluorinated ethylene propylene, ceramics and combinations thereof.

In a preferred embodiment, the spots can project inwards into the chamber from about 0.01 mm to about 10 mm, preferably from about 0.5 mm to about 5 mm, more preferably from about 0.1 mm to about 2 mm, so that direct exposure to microwaves is restricted to the lower portion of the sample tube.

In another embodiment, the chamber can display cuboid shape with a length from about 40 to about 60 cm, a width from about 8 cm to about 12 cm, and a height from about 4 cm to about 8 cm.

In a further embodiment, a plurality of chambers with a plurality of stationary spots located longitudinally can be connected to each other. The electromagnetic radiation can propagate into the plurality of chambers through a plurality of slits or openings on the walls of said chambers. In another embodiment the chamber can display circular shape and said stationary spots are located circularly along the chamber. In a preferred embodiment, the chamber is made of microwave- reflecting material such as metal, aluminium, stainless steel, or combinations thereof. In another embodiment, the apparatus is operated at atmospheric pressure.

According to another aspect of the invention, the microwaves are generated with a solid state microwave generator (SSMW), in other words with a generator that generates the microwaves by power semiconductor devices and not by a magnetron tube. The electromagnetic radiation generated by the semiconductor generator may be advantageously introduced to the chamber by a coax connection and an antenna.

The disclosure further relates to a method of isolating nucleic acids for subsequent analysis by a DNA polymerase chain reaction encompassing the conversion of electromagnetic radiation into thermal energy, comprising the steps of: i) providing the apparatus according to the disclosure; ii) providing a plurality of sample tubes with closable means which are detachably connected to the plurality of stationary spots; and loading each sample tube with a) a sample to be analysed, b) a tablet comprising water insoluble hydrated magnesium silicate and crystalline phosphate buffer saline, and c) water to obtain a weight ratio of tablet to sample in the vessel from 1:5 to 5:1; and closing the vessel; iii) placing each sample tube into each thermally- conductive stationary/immobile spots; iv) operating the microwave generator so that an standing microwave is generated, conveyed through the chamber and contacting the plurality of stationary/immobile spots; v) propagating microwave radiation through the plurality of stationary/immobile spots into the plurality of sample tubes, whereby thermal energy is generated, so that a temperature rise up in the range from 85° C. to 140° C. of the mixture inside the closed vessel is elicited and nucleic acids are released; and vi) optionally separating the water-insoluble components adsorbed on the magnesium silicate and removal of the supernatant containing soluble nucleic acids, followed by desalting to obtain a solution of nucleic acids suitable for PCR analysis.

The disclosed method may further comprise the steps of dissolving the buffer components of the tablet to obtain an aqueous phosphate buffered saline solution having a pH from 5.5 to 7.0 and a salt concentration of 0.4 to 1.2 mol/L; and operating the microwave generator for about 5 seconds to 2 minutes, preferably for about 10 seconds to 1 minutes, most preferred for about 15 to 30 seconds.

Another embodiment relates to the use of a tablet for extracting nucleic acids from a sample in a method according to the disclosure, wherein the tablet is a non-aqueous mixture of solids comprising from 30 to 70 percent by weight crystalline phosphate buffer saline; and from 10 to 40 percent by weight water insoluble hydrated magnesium silicate particles.

In another embodiment, the disclosure relates to the use of a tablet for extracting nucleic acids from a sample in a method according to the disclosure, wherein the tablet for extracting nucleic acids further comprises from 15 to 45 percent by weight hydrophilic colloid, wherein the hydrophilic colloid is cellulose, carboxy-methyl cellulose, cellulose derivatives, alginate, starch, xantan gum, arabic gum, guar gum or mixtures thereof.

The apparatus, method and tablet according to the disclosure can be 20 used for isolating and characterizing nucleic acids from raw and/or processed animal and plants materials and processed products thereof; allergens present in cereals and products thereof, chickpea and products thereof, casein, almond and products thereof, cashew and products thereof, peanut and products thereof, hazelnut and products thereof, macadamia and products thereof, mustard and products thereof, soya and 25 products thereof, sesame and products thereof, walnut and products thereof, pistachio and products thereof, lupin and products thereof, celery and products thereof, fish and products thereof, crustaceans and products thereof; genetically modified organisms such as genetically modified maize, soy beans, rape seed, potatoes, tomatoes; animal material such as horse, pig, sheep, poultry; plant material such as apricot kernels, 30 cherries, peaches; pathogens such as viruses bacteria; Salmonella spp., Listeria spp. Shigella spp., Campylobacter spp., Cronobacter, Clostridium spp., Legionella spp., Enterobacteriaceae, Escherichia spp; human and veterinary samples such as blood, faeces; and forensic samples such as swabs.

According to the present disclosure, there is no longer a requirement for 35 the addition of surfactants, enzymes, detergents, organic solvents, etc. for dissociation of a complex matrix. Temperatures up to 140° C. and high osmotic strength, due to the high salt concentration, cooperate to dissociate all cell structures and, thus, to promote a release of nucleic acids from the cells into the aqueous solution. The buffering agent contained in the tablet is responsible for creating an osmotic shock, which forces the cytoplasm and, in particular, the cell nucleus to release its content into the extraction solution. The combination of osmotic pressure and short exposure to high temperatures enhances the preservation of nucleic acid integrity for subsequent analysis. The nucleic acids strands are further stabilized by the acidic phosphate buffer, even in aqueous solution with temperatures up to 140° C. This method can be used with virtually all complex matrices, such as food, feed, human, veterinary and forensic samples, enabling an efficient extraction of nucleic acids from nearly all biological matrices. Efficient extraction means that i) the isolated nucleic acid solution is free of DNA polymerase specific inhibitors and, thereby, ii) the subsequent polymerase chain reaction (PCR) can be performed faster and with higher degree of reproducibility than conventional methods.

By way of the present disclosure, the temperature of aqueous solutions confined in closed conventional (screw cup) vessels, which are exposed to microwaves, can be tightly controlled. Until now, the rise of temperature in small volumes with the aid of microwaves was unpredictable. The combined used of a single tablet and the apparatus according to the disclosure yields high DNA quality, i.e. free of PCR inhibitors, and reduces extraction time to the range of seconds. An important advantage is that the temperature of the aqueous solution is rapidly raised by microwave-mediated heat transfer without the disadvantage of vessel bursting due to uncontrolled increase of vapor pressure inside the vessel. This also allows the processing of several samples simultaneously without cross-contamination and spillage of the vessel's content.

Advantageous is the straightforward establishment of a routine procedure for the simultaneous analysis of a variety pf samples regardless of their different physico-chemical properties. Such samples can be animal and plant foodstuffs rich in lipids, polysaccharides, proteins, and, in particular, mixtures thereof. There is no need for experienced personnel or additional equipment. Any person with minor laboratory experience is, thus, able to carry out an efficient extraction of nucleic acids from any biological sample, even when handling complex matrices. Full standardization of nucleic acid extraction is provided in an unprecedented short-term by way of the disclosed apparatus and method.

It is assumed that the disclosed microwaving-step leads to disruption of cellular structures and denaturation of peptidic DNA polymerase specific inhibitors (herewith also referred to as “molecular digestion”). Further inhibitors with hydrophobic, lipophilic and acidic properties and, at least, a portion of lipids, proteins, polysaccharides and salts contained in the matrix become adsorbed on the magnesium silicate particles. Subsequent removal of the precipitated material from the aqueous fraction containing the genomic DNA, which can be subsequently desalted, enables the provision of a PCR inhibitor-free DNA solution within a fraction of the time periods needed following conventional procedures. Desalting can be performed by size exclusion chromatography, ultra-filtration or conventional DNA binding chromatography. Optionally, the PCR sample may be simply diluted, preferably in a ratio from 1:5 to 1:10, so that the salt concentration is lowered to appropriate levels.

It is well known that microwaves are heterogeneously distributed within the cooking chamber of conventional microwave ovens, which display a multimode pattern of microwave spreading. This means that unpredictable node/internode microwave patterns are propagated throughout the cooking chamber. As a result, some areas have higher exposure to electromagnetic energy than others and, thus, differential heating occurs depending on the spot within the oven. In order to partially compensate these differences, most ovens exhibit rotating means. Rotating a closed vessel loaded with an aqueous solution does not avoid vessel bursting when exposed to microwaves. In other words, the targeting of a sample with the precise amount of energy to reach up to 140° C. and, thus, to achieve a homogeneous dissociation of the cellular and sub-cellular components cannot be effected in conventional chambers such as microwaves ovens, which are arranged to generate a multimode microwave pattern.

It is assumed that, because of the material, shape and dimensions of the disclosed chamber, microwaves may adopt a standing wave in monomode pattern within the chamber. It appears that the location of the stationary spots with respect to the maxima of the standing wave enables controlled homogenous heating of the samples located at the stationary spots; to a certain extent, irrespective of the irradiation time. Moreover, the position of the bottom part of the sample tubes projecting with a predetermined length into the chamber appears to influence both the overall power distribution within the chamber and the transfer of energy into the sample mixture. It seems that, due to the specific arrangement of the sample tubes within the apparatus, the electromagnetic energy is coupled by the tubes in a way that efficient heating of the sample mixture is achieved, while avoiding an excessive heating of the magnetron, which may be back irradiated by the standing way. By means of the present apparatus, the adverse influence of microwaves propagating back to the magnetron is efficiently reduced and, thus, any damage to the microwave source is minimized. Further, such a fast and homogenous heating leads to an evenly dissociation of the cellular components of the sample. This effect is assessed by a resulting quick and efficient DNA extraction, and lack of vessel bursting after microwaving. Notably, the effect conferred by the use of the disclosed apparatus can only be achieved in combination with the disclosed tablet comprising a non-aqueous mixture of solids. The amount of salt added to the sample by way of the disclosed tablet appears to alter the dielectric heating pattern in the aqueous solution, leading to a faster molecular rotation and, thus, faster heat-transfer to the sample solution. The insoluble magnesium silicate contained in the tablet cooperates with electromagnetic radiation by absorbing energy in form of heat and, subsequently, by transferring this energy into the aqueous solution progressively. This, in turn, favors an evenly distribution of energy within the liquid which prevents bursting due to a sudden rise of vapor pressure. Thus, the synergistic action of salt in solution, insoluble magnesium silicate and exposure to microwaves in accordance with the apparatus and method of the disclosure eliminates the sudden explosive behavior of liquids confined in sample vessels exposed to microwave radiation, even at longer periods of irradiation.

Evaluation of the quality of the extracted nucleic acids and/or the presence of inhibitors in a sample to be analysed is performed based on the Ct value obtained by quantitative real-time polymerase chain reaction. Fewer cycles necessary to amplify target DNA extracted from the sample are illustrated by lower Ct values. In other words, low Ct values indicate low amount of PCR inhibitors present in the sample and, thus, an efficient DNA extraction. The apparatus, method and kit of the disclosure allows for fast, reliable and economically advantageous nucleic acid extraction from complex matrices without the disadvantages of conventional approaches.

The conventional methods require an exhaustive removal of added surfactants, reagents and/or solvents to avoid inhibition of the polymerase chain reaction. Also biological samples are known to contain inhibitors. Typical PCR inhibitors endogenous to biological samples are collagen, myoglobin, hemoglobin, immunoglobins and heme (meat and blood), complex and other polysaccharides (feces, plant materials), humic acid (soil, plant materials), melanin and eumelanin (hair, skin), calcium ions and proteinases (milk, bone), and bile salts (feces). The present disclosure overcomes the problems associated with endogenous and added PCR inhibitors. Firstly, expensive surfactants or reagents are not required, and, second, removal of inhibitors is provided by binding to hydrated magnesium silicate particles, which are supplied by the tablet. Further, the method has the advantage that it can be applied simultaneously to very different complex matrices, and allows for the isolation of DNA from different species with the same workflow.

Further advantages, goals and embodiments of the invention can be understood from the following detailed description of examples and the drawings. The invention shall not be limited to the examples, but it has been defined in the claims.

### BRIEF DESCRIPTION OF THE DRAWINGS

In the drawings:

FIG. 1 is a graphic representation of the electric field distribution (V/m) at P=1 kW within the chamber by computer simulation.

FIG. 2 is a graphic representation of the power dissipated within the sample tube (W/cm3) at P=1 kW by computer simulation.

FIG. 3 is a graphic representation of the power dissipated within the content of the sample tube (W/cm3) at P=1 kW by computer simulation.

FIG. 4 is a schematic representation of the apparatus representation of the longitudinal section of the apparatus with sample tubes according to the disclosure.

FIG. 5 is a schematic representation of the cross section of the apparatus with sample tubes according to the disclosure.

FIG. 6 is a schematic representation of the top view of the apparatus with sample tubes according to the disclosure.

FIG. 7 is a schematic representation of interconnected chambers according to the disclosure displaying slits or openings.

FIG. 8 is a schematic representation of the top view of an apparatus according to the disclosure having circular shape.

FIG. 9 is a schematic representation of an apparatus according to the disclosure with a plurality of stationary spots.

FIG. 10 shows the relation between the impulse mode of the magnetron and the temperature within the sample tube over time.

FIG. 11 shows a rectangular chamber according to the disclosure that is fed by a semiconductor generator.

FIG. 12 shows a chamber having the form of a circular tube that is fed by a semiconductor generator.

FIG. 13 is a block diagram showing a possible design of a semiconductor microwave generator.

### DETAILED DESCRIPTION OF THE INVENTION

By way of the disclosed apparatus and method, nucleic acids are isolated from a complex matrix, such as a food sample, for further analysis by DNA polymerase chain reaction. According to the disclosure, the cells of the sample are lysed in aqueous solution, whereby the process encompasses subjecting the sample to microwave-mediated heat transfer. Microwaves are non-ionizing electromagnetic waves with frequencies ranging from 300 MHz up to 300 GHz, and wavelengths λ from 1 meter to 1 millimeter, respectively. Magnetrons are devices able to generate microwaves, which are distributed differently depending on the material and shape of the cavity through which the microwaves propagate. For example, microwave frequencies of 2.45 GHz, which corresponds to a wavelength of 12.23 cm, are generated by magnetrons in conventional microwave ovens.

Microwave-reflecting materials, such as aluminium, are those which reflect incident microwave energy. In other words, electrically conducting metals heat up marginally because they have high thermal conductivity and do not absorb the generated microwave field. On the other hand, microwave-transparent materials such as plastic are not electrically conductive and do not reflect microwaves. Thus, microwave-transparent materials interact marginally with the microwaves and do not alter the wave pattern within the microwave cavity.

A cavity resonator is a hollow closed conductor such as a metal box or a cavity within a metal block, containing electromagnetic waves reflecting back and forth between the cavity's walls. When a source of microwaves at one of the cavity's resonant frequencies is applied, the oppositely-moving waves form standing waves, and the cavity stores electromagnetic energy. In a monomode microwave system, a standing wave pattern is generated due to interfering fields of the same amplitude but with different oscillating directions. An array of nodes, where microwave energy intensity is 0, and antinodes, at which microwave energy is at its highest, is produced in a monomode microwave pattern. In a multimode microwave system, such as conventional microwave ovens, no standing wave is generated and chaotic microwave dispersion takes place. In other words, the energy density distribution cannot be predicted in multimode microwave systems.

The apparatus according to the disclosure may comprise a cavity resonator in form of a cuboid chamber having following dimensions: length from about 40 to about 60 cm;

width from about 8 cm to about 12 cm; and height from about 4 cm to about 8 cm. The chamber can also have a circular shape. The chamber is made of a microwave-reflecting material such as metal, aluminium, or alloys thereof, having a wall thickness from about 1 to about 5 mm.

The apparatus according to the disclosure displays stationary spots arranged for receiving sample tubes. The stationary spots are fixedly arranged on the upper part of the chamber. The chamber displays circular openings at the location of such spots. Said spots may have cylindrical shape having open ends. The cylinders can be made of microwave-reflecting material, such as aluminium, and they are fixedly attached to the chamber by soldering the lower edge of cylinder to the upper side of the chamber. Thereby, the stationary spots represent an interface between the inner side of the chamber and the outside area. The microwaves propagating within the chamber cannot escape through the spots because their openings have a diameter smaller than the wavelength of the microwaves. The stationary spots may display the following dimensions: diameter from about 0.5 to about 2.5 cm; height from about 0.5 cm to about 3 cm.

According to the disclosure, the stationary spots can comprise means for holding sample vessels. The disclosed holding piece has a cylindrical shape with open upper end and closed bottom end. The upper edge or rim extends from about 1 to 15 mm over the edge of the metal cylinder at the stationary spot, so that it cannot fall into the chamber. The holding piece is located at the inner side of the stationary spot in intimate contact with the metal cylinder and fits tightly. The holding piece made of a microwave-transparent material such as silicon, ceramic, silicon carbide, resin, Teflon®, polytetrafluoroethylene, perfluoroalkoxy, fluorinated ethylene propylene and combinations thereof.

The apparatus according to the disclosure displays an electromagnetic radiation generator, such as a magnetron, at one end of the chamber. The magnetron may generate microwaves of wavelengths from about 5 cm to about 30 cm. The chamber displays one opening close to the magnetron, through which the generated microwaves enter the chamber. The magnetron may be operated at a frequency of about 6 GHz to about 1 GHz, at a power from about 100 W to about 1500 W. An energy absorber may be positioned at the opposite end of the chamber (with respect to the magnetron's position) so that an excess of energy in form of heat can be removed from the chamber.

The stationary spots according to the disclosure are arranged along the upper part of the chamber and they are essentially aligned with the magnetron. The spots are located longitudinally at intervals or gaps from about 3 cm to about 15 cm, preferably from about 4 cm to about 13 cm, most preferred from about 5 cm to about 11 cm. The chamber can have a circular shape and the stationary spots can be located circularly along the chamber. The number of stationary spots is only limited by the size of the chamber.

In one embodiment, the stationary spots display antennas projecting into the chamber for coupling of the microwave energy.

According to the present disclosure, the sample tubes are made of a microwave transparent material such as plastic. The sample tubes can be introduced into the holding piece so that they are detachably but tightly connected to the stationary spot. According to the disclosure, the edge of the bottom part of the tube project into the chamber from 0.01 mm to 10 mm, preferably 0.1 mm to 5 mm, more preferably 0.5 mm to 2 mm. In other words, only a fraction of the bottom part of the sample tube projecting into the chamber is directly exposed to the standing microwaves. This arrangement favors a homogenous energy distribution of the electromagnetic field within the chamber, so that fast but progressive transfer of energy into the sample mixture is achieved. By means of such positioning and the promoted interaction with the standing microwaves, an optimum coupling of the electromagnetic energy is achieved. Such coupling favors, in turn, a reduction of excessive power reflected back to the magnetron, thereby prolonging its operation life.

The sample tubes of the disclosure can be inexpensive conventional screw cup plastic vessels. The sample tubes can display a bottom part with an inner conical end and an outer cylindrical wall separated by a gap. Without been bound by theory, it seems that the presence of such vessel arrangement favors a better energy transfer than with vessels having a flat bottom without a gap.

According to the method of the present disclosure, the sample to be analysed can preferably be mechanically dissociated into particles, dispersion or solution. A portion of the sample ranging from 50 mg to 500 mg can be weighted and loaded into a vessel made of microwave-transparent material having closable means, such as a screw cap or safe lock. A predefined amount of a composition for extracting nucleic acids may be pressed or compressed to obtain a tablet; in this form, it may be added to the dissociated sample. Said tablet may be a mixture of solids comprising from 30 to 70 percent by weight crystalline phosphate buffer saline, and from 10 to 40 percent by weight water insoluble hydrated magnesium silicate particles. The phosphate buffer salt may be present in the mixture of solids as fine crystals or in granulated form, and its composition is preferably as follows: NaCl 137 mmol/L, Na₂HPO₄⋅2 H₂O 10 mmol/L, KGl 2.7 mmol/L, KH₂PO₄ 2 mmol/L. The phosphate buffer salt gives a hypertonic solution after dissolving in water, forcing a release of nucleic acids from the biological sample through the resulting osmotic shock. The water-insoluble hydrated magnesium silicate is preferably a hydrated magnesium silicate or fine talc in the form of powder or fine granules. Said hydrated magnesium silicate powder may have a median particle size in the range of 1.0 to 2.0 pm, preferably from to 1.5 pm; a median diameter D50 in the range of 0.8 to 2.5 pm; and a density of 2.6 to 2.8 g/cm³. The insoluble silicate powder has a large surface for adsorption of lipids, complex polysaccharides and other potential polymerase inhibitors. Said tablet may further comprise from 15 to 45 percent by weight swellable hydrophilic colloid, which can be selected from cellulose, carboxy-methyl cellulose, cellulose derivatives, alginate, starch, xanthan gum, arabic gum, guar gum or mixtures thereof. The swellable hydrophilic colloid can both facilitate the compacting of the composition as well as the dispersion of the mixture of solids upon contact with an aqueous solution. The swellable material must be free of contaminants, in particular plant and animal nucleic acids, genetically modified organisms and allergens. A predefined amount of composition in form of a tablet may be added so as to obtain a weight ratio of tablet to sample in the vessel from 1:5 to 5:1.

An amount of water may be added to the dissociated sample and tablet in the vessel to dissolve the buffer components and to obtain an aqueous phosphate buffered saline solution having a pH from 5.5 to 7.0 and a salt concentration of 0.4 to mol/L. After closing the vessel, a vortexing or shaking step may be carried out from 1 to 120 seconds for proper dissolution of the tablet's components, followed by a short spin-down to avoid accumulation of the dispersion of sample and tablet at the lid of the vessel.

In one embodiment, upon operation of the apparatus according to the disclosure, the temperature inside the sample vessel containing i) sample, ii) composition for DNA extraction and iii) water, can be raised in the range from 85° C. to 140° C., preferably from 90° C. to 130° C., most preferred from 95° C. to 120° C., without bursting of the sample tube and spillage of the sample.

Computer simulations of the energy distribution within the chamber and the samples were generated with the software COMSOL Multiphysics considering following parameters: i) length of the chamber, ii) position of the magnetron, and iii) horizontal and iv) vertical position of the samples.

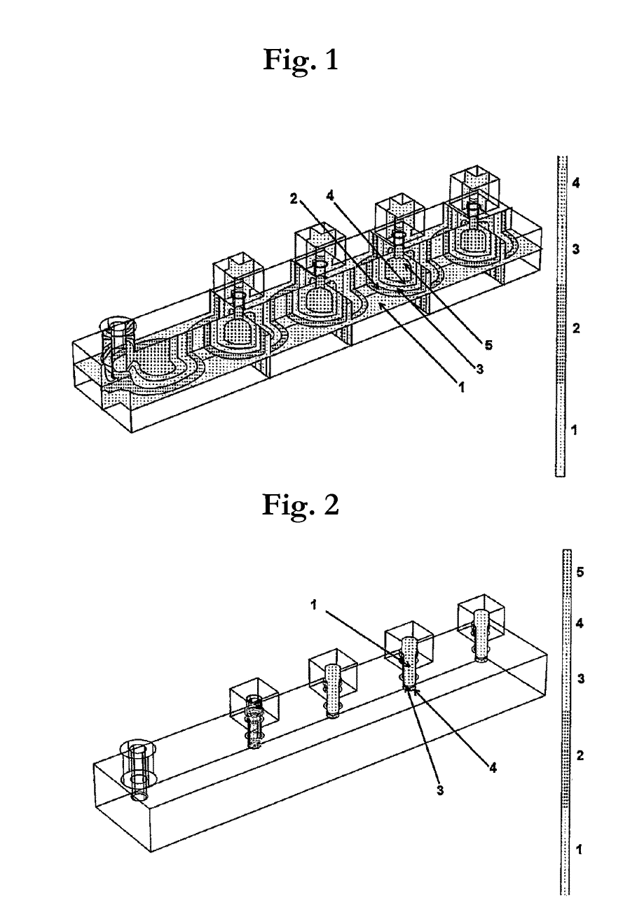

FIG. 1 shows a computer simulation of the electric field distribution (V/m) within a chamber according to the disclosure, whose dimensions allow for the formation of a homogenous monomode microwave pattern upon operation of the magnetron at 1 kW. The power of 1 kW is only a representative example. Of course, the chamber of the invention may be operated with any other power value suitable or necessary to heat the samples. The power level only influences the amplitude of the standing wave but not the important quantity, that is the spacial distribution. Depicted is a monomode pattern of microwave energy distribution, with highest energy at regular intervals maxima/internodes of the standing wave. An electromagnetic energy gradient (lowest 1 to highest 5) is established within the chamber. As will be understood by a skilled person the electrical field strength and accordingly the electromagnetic energy density rises when approaching the location where the sample tubes have been fitted into the chamber, say from the inside of the chamber (see for example the scale values 1 to 5 in FIG. 1 at the site of the second sample tube from the right). The electromagnetic energy density reaches its maximum value at the location of the sample tubes such that the samples are heated fast and effectively.

FIG. 2 shows a computer simulation of the power dissipation (W/cm³) throughout sample vessels and holding pieces placed onto chamber according to the disclosure, whose dimensions allow for the formation of a homogenous monomode microwave pattern upon operation of the magnetron for example at a power of 1 kW. An electromagnetic energy gradient (lowest 1 to highest 5) is established within the sample tubes. Marginal energy dissipation is found throughout the plastic material of sample vessel and the holding piece. This is necessary and desirable to avoid excessive power loss and damaging (melting, degradation) of the plastic material of the sample tubes and holding pieces. FIG. 2 shows a minor coupling of microwave energy by the sample vessel. As seen the maximum power density appears (4 in FIG. 2 at the second sample tube from the right) in a narrow region closely at the bottom of the sample tube and rapidly decreases in the upper portion of the sample tube.

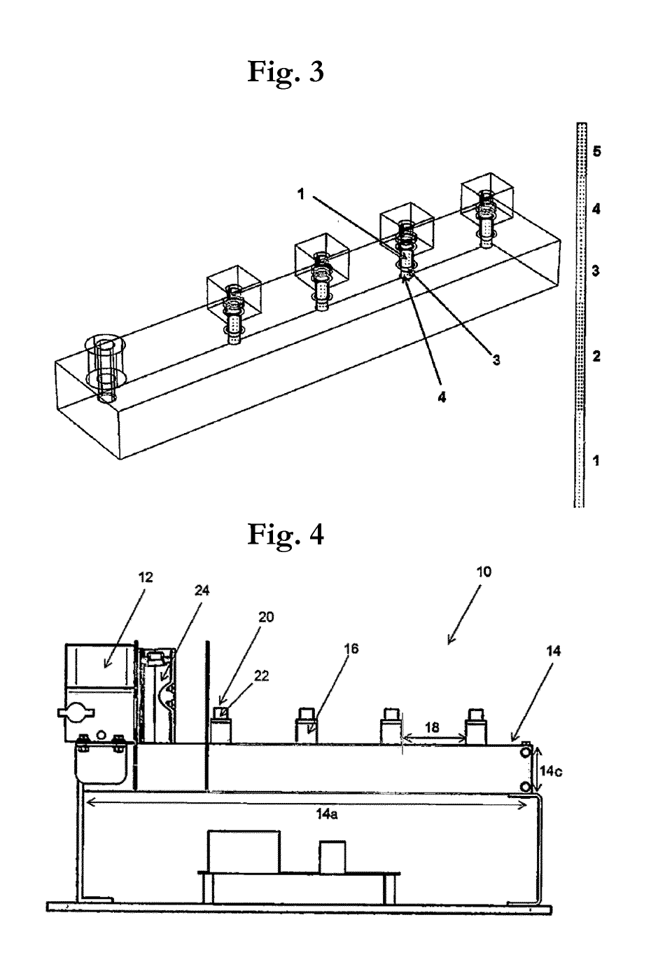

FIG. 3 shows a computer simulation of the power dissipation (W/cm³) throughout a sample, comprising a substance, such as water, able to be heated by dielectric heating, and placed onto the chamber according to the disclosure, whose dimensions allow for the formation of a homogenous monomode microwave pattern upon operation of the magnetron for example at a power of 1 kW. An electromagnetic energy gradient (lowest 1 to highest 5) is established within the sample. It is apparent that the content at the bottom of the sample tube is more exposed to microwave energy than the content in the upper part of the sample tube. An electromagnetic energy gradient is generated, displaying highest energy coupling to the content at the bottom of the tube, and lowest coupling to the content at its upper part (see the scale values 1, 3 an 4 at the second sample from the right in FIG. 3).

The disclosure aims at the establishment of a homogeneous distribution of microwave energy throughout the sample mixture to avoid sudden explosive behaviour and, thus, sample loss and spillage.

Notably, the effect conferred by the use of the disclosed apparatus can only be achieved in combination with the disclosed tablet. The amount of salt added to the sample by way of addition of the tablet appears to alter the dielectric heating pattern in the aqueous solution, leading to a faster molecular rotation and, thus, faster heat-transfer to the sample solution. Non-soluble magnesium silicate particles cannot be solubilized and slowly precipitates at the bottom of the sample tube after mixing. The insoluble magnesium silicate added to the sample cooperates with electromagnetic radiation by absorbing energy in form of heat. This energy is, then, progressively transferred into the aqueous solution and distributed by convection in the liquid which, in turn, favors an even distribution of energy within the liquid preventing bursting due to the sudden rise of vapor pressure. Thus, the synergistic action of salt in solution, insoluble magnesium silicate and exposure to microwaves in accordance with the apparatus and method of the disclosure reduces the sudden explosive behavior of liquids exposed to microwave radiation, even at longer periods of irradiation, while obtaining DNA samples free of PCR polymerase inhibitors.

Numerical analysis of the power balance in the simulation shows the following power distribution among the different components of the apparatus and sample vessels. The highest amount of the energy (92.81%) is reflected as a standing wave within the chamber, propagating back and forth, which according to the present disclosure displays stable field maximas, as shown in FIG. 1, at which the sample tubes and holding pieces are positioned. The substance inside the sample vessel subjected to dielectric heating absorbs 6.07% of the total generated energy (FIG. 3), while the metal walls of the chamber absorbed 1.07%. The sample vessels and the holding piece, with absorption rates of respectively 0.05% and 1.1 x 10′6% of the total power (FIG. 2), are the components at which the power distribution is the lowest. A marginal power leakage represents 1.9×10⁻⁶% of the total power.

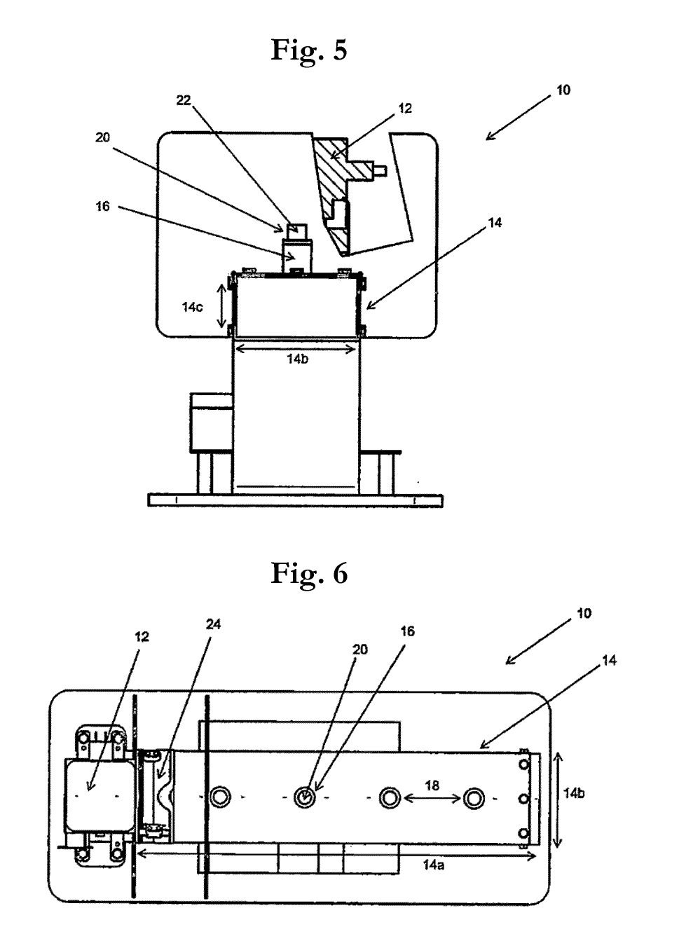

FIG. 4 is a schematic representation of the longitudinal section of the apparatus 10 with sample tubes 20 according to the disclosure. The apparatus 10 comprises a chamber 14, whose dimensions 14a, 14b (see FIGS. 5) and 14c allow the generation of a standing wave upon operation of the magnetron 12. Sample tubes 20 are closed by means of screw cups 22 and placed into the stationary spots 16, whereby they are exposed to microwave radiation in monomode pattern. The stationary spots 16/sample tubes 20 are placed at regular intervals 18 of predetermined length corresponding to the microwave maxima and therefore the maximum power density of the standing wave (see FIG. 1). A cooler unit 24 is located close to the magnetron for cooling during operation. The apparatus according to the disclosure can be controlled by electronic means that may be located below the chamber (an example is depicted below reference number 14a in FIG. 4).

FIG. 5 is a schematic representation of the cross section of the apparatus 10 with sample tubes 20 according to the disclosure seen from the side of the magnetron 12 (left side in FIG. 4). For a better visibility the magnetron 12 is only depicted partially (at the reference number 12). Otherwise only contour lines are shown such that the parts behind the magnetron in the viewing direction are not hidden. It can be observed that the stationary spots 16/sample tubes 20 are positioned in the middle part of the chamber 14 and they are essentially aligned with the magnetron 12. Because of the dimensions 14a, 14b, 14c of the chamber 14 (see FIG. 4 and FIG. 5) and the highly conductive walls of the chamber 14, a standing microwave in monomode pattern can be generated (see FIG. 1). The maximum power densities of the microwave pattern precisely meet the sites at which the sample tubes are fit in the chamber 14. In this way, the samples are heated optimally.

FIG. 6 is a schematic representation of the top view of the apparatus 10 with sample tubes 20 according to the disclosure (viewing direction from the upper side in FIG. 4). It is apparent that the stationary spots 16 are located at regular intervals 18 of predetermined length; and they aligned with the magnetron 12. In this view the equal distances 18 of the sample tubes that correspond to the power density maxima of the standing microwave are shown clearly.

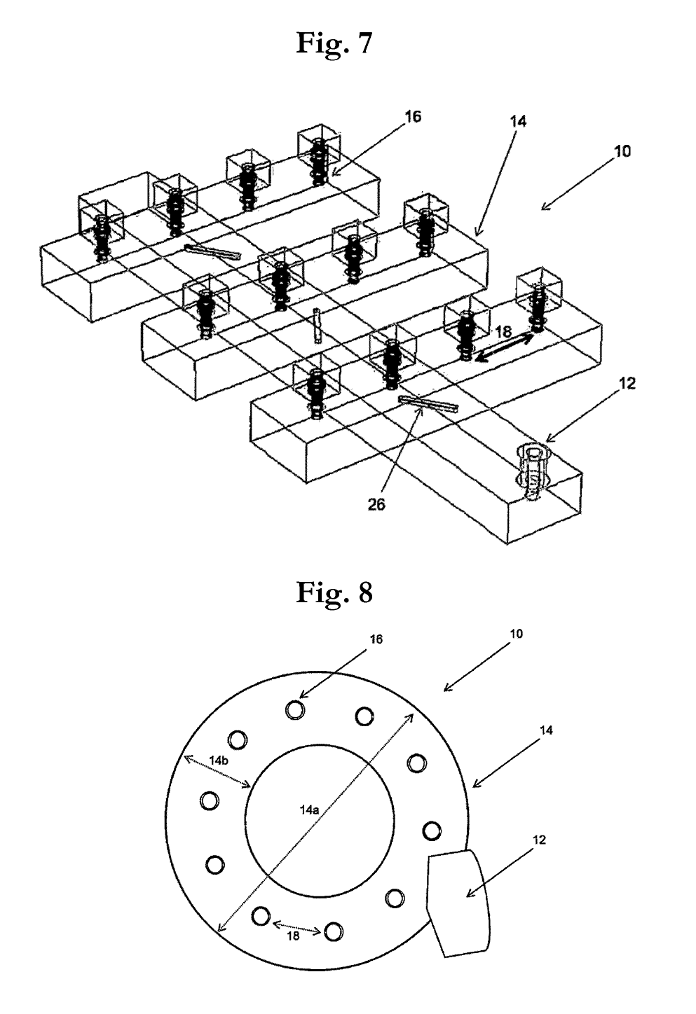

See FIG. 7; in one embodiment, the apparatus 10 can display a plurality of chambers 14 connected to each other having a plurality of stationary spots 16 located longitudinally in the respective chambers. The electromagnetic radiation propagates into the plurality of chambers 14 through a plurality of slits or openings 26 on the walls of the connected chambers 14. The chambers 14 in FIG. 7 are depicted with identical dimensions and the same number of stationary spots for the sample tubes. However, it is not necessary to use identical dimensions or the same number of stationary spots as long as a standing wave having maxima at the sites of the sample tubes is present in every chamber 14 because of the dimensions of the chambers, the coupling and the frequency of the microwave. Further, the chambers 14 do not have to be parallel to each other at all.

See FIG. 8; in a further embodiment, the chamber 14 of the apparatus 10 displays circular shape, having a diameter/length 14a and chamber width 14b, whose dimensions favor the propagation of microwave radiation within the chamber 14 as a standing wave. The stationary spots 16 are preferably located circularly along the circular chamber 14, for example on a concentric circle line in the middle between the two circular chamber walls. The stationary spots are separated from each by identical intervals 18 of predetermined distance. The magnetron 12 is connected to the chamber 14 sideways at its outer wall. Besides the linear and circular chambers 14 described above by way of example any other chamber geometries are allowable as long as a standing wave may be established having the sample tubes in its maxima.

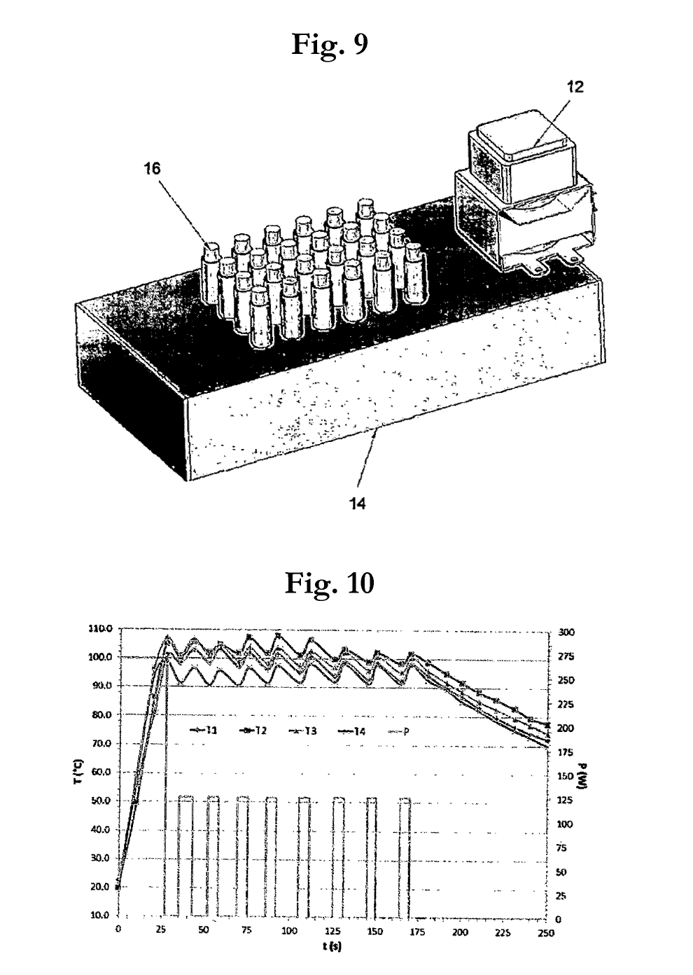

See FIG. 9; in one embodiment, the apparatus 10 may exhibit a plurality of stationary spots 16, for example, 24 spots. In the above embodiments, there is only a single holding piece or a single sample tube in every maximum of the standing wave in the chamber 14, or in other words, there is only a single holding piece after every interval 18. In the contrary, in this embodiment, after every interval 18 there is a number of sample tubes on a straight line perpendicular to the interval direction (in FIG. 9, there are four sample tubes on straight lines from the upper left side to the lower right side). The four sample tubes on every straight line and the 24 sample tubes in FIG. 9 are just shown as examples. Any other numbers are possible as long as it is ensured that every sample tube is within a maximum of the standing wave because of the dimensions of the chamber 14 with regard to the frequency of the microwave. If this holds, as shown in the circular embodiment of FIG. 8, more than one concentric circles with sample tubes are possible.

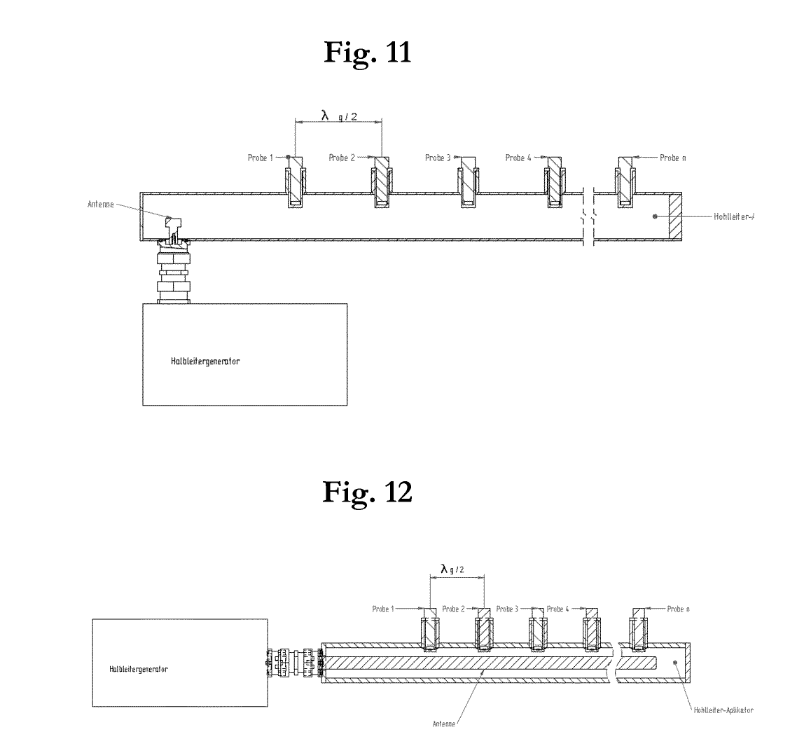

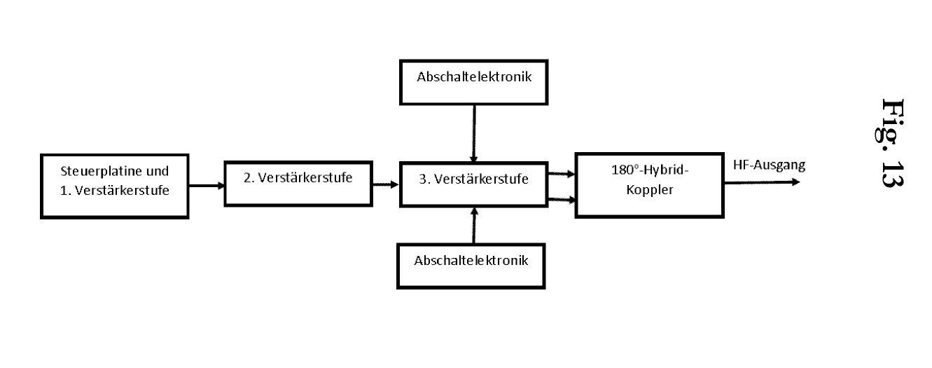

FIG. 11 shows a chamber 14 according to the invention that is fed with microwave energy by a semiconductor generator (SSMW) using a coax line and an antenna in the chamber. FIG. 12 is an embodiment in which the chamber 14 has the form of a circular tube. The circular tube chamber is also fed with microwave energy by a semiconductor generator using a coax line and an antenna .

The benefits of an SSMW generator are obvious, because in such a device there is no need for a magnetron and a high voltage of some thousand volts to operate the magnetron. In a laboratory the spillage of greater amounts of inflammable or electrically conductive liquids always has to be faced. Bottles may brake, cocks and hoses may leak, chemicals may sublime or creep and deposit at some places over the atmosphere. This may damage high voltage parts in laboratory devices (fire hazard) and lead to electrical dangers for the laboratory personnel. Therefore, it is desirable not to use high voltages if possible.

Further, the power of the SSMW generator may be automatically adapted to the absorbed or transmitted power. The power output may also be adjusted by hand without limitations. At the moment, a power up to 1 kW is possible. The SSMW generator may be operated such that it detects the reflected power on its own and switches off itself if the reflected power is greater than an upper lever. Further, the design may be more compact. The SSMW generator may be used in two operating modes, will say a continuous mode and an impulse mode. In the impulse mode the generator creates less heat.

FIG. 13 is a block diagram showing a possible design of a semiconductor microwave generator. As can be seen the semiconductor microwave generator mainly comprises three amplifier stages. The third amplifier stage has two outputs that emit power. The 180° hybrid coupler combines the power of both outputs and transmits it to the RF output. Both shut-down electronics (depicted over and under the third amplifier stage in FIG. 13) for the two power branches of the third amplifier stage may be used to switch off the generator if the load—as described above—reflects to much power.

According to the present apparatus 14 and method, an increase in the temperature of the sample is achieved by microwaving the mixture of sample, DNA extraction tablet and water contained in the sample tubes 20, which are located onto 20 said chamber 14. The microwaving step can be applied for about 5 seconds to 2 minutes, preferably for about 10 seconds to 1 minute, most preferred for about 15 seconds to 30 seconds. The microwave generator 12 may be operated at a power from 100 to 1000 Watt, preferably 125 to 600 Watt, more preferred 150 to 300 Watt. The microwave electromagnetic radiation elicits a temperature rise of the mixture inside the 25 closed vessel 20 up to 140° C. After the microwaving step, the sample tube 20 may optionally be vortexed to further the homogenization of the heated mixture. The synergistic activity of the apparatus 10 and the DNA extraction tablet allows for fast temperature increase without solution bursting and, thus, an efficient release of nucleic acids; the integrity of the vessel 20 and sample is thereby not compromised. The 30 stability of the extracted DNA, assessed by subsequent PCR analysis, is preserved even if treated at 140° C. for long periods because of the salt and phosphate provided by addition of the tablet. A release of nucleic acids occurs when cellular structures (membranes, organelles, etc . . . ) are so disrupted that no interaction of nucleic acids with any proteins, lipids and polysaccharides takes place. A release of the nucleic acids 35 from the cell nuclei is effected through a large osmotic difference created by the hypertone solution. The rapid increase in temperature leads to efficient denaturation of proteins and disruption of cell walls. Also, an increased solubilisation of lipids and polysaccharides is promoted. These cellular components are further adsorbed and precipitated through binding on the water-insoluble magnesium silicate particles, reducing the binding to the inner walls of the sample tube (20) which, in turn, eliminates subsequent carry over into the analytical steps. It appears that the magnesium silicate is very effective in absorbing microwave energy and transferring it into the aqueous solution, preventing solution bursting and sample spillage. The water-insoluble components adsorbed on the magnesium silicate may be separated from the aqueous phase by centrifugation or filtration. The aqueous supernatant or filtrate containing soluble nucleic acids may be desalted or diluted to lower the salt concentration so as to obtain a solution of nucleic acids suitable for PCR analysis. Desalting can be carried out by affinity chromatography such as commercial silica-based nucleic acid extraction columns, size exclusion chromatography or ultra-filtration.

The apparatus 10 and tablet for extracting nucleic acids according to the disclosure solve the problem of efficient heating and DNA extraction without solution bursting and explosive behavior. First, the high salt concentration promotes a faster molecular motion within the aqueous solution under the electromagnetic field and, thereby, facilitates dielectric heating. This results in a rapid heat-up of the water-containing sample. It is important to keep the bottom part of the tube 20 below the top edge of the chamber 14, so that the sample is subjected to the standing microwave (see FIG. 1) generated within the chamber 14. It has been ruled out that homogenous sample heating, which ultimately allows for a fast and efficient DNA extraction, is cooperatively dependent on i) the amount of salt and magnesium silicate contained in the tablet, ii) the position of the stationary spots relative to the wavelength of the irradiated microwaves, and iii) the length of the sample tubes projecting into chamber. This effect is highly significant when a sample in aqueous solution is confined in a closed vial. It is known that water contained in a sample may be heated up by exposure to microwave radiation. However, the heating process of small volumes is rather long and unpredictable. Fine control of the increasing temperature without the risk of bursting of the vessel was, until now, not possible. This is, in part, due to the uneven vapor pressure distribution within the aqueous solution. This phenomenon is characterized by the typical sudden explosion of liquids and foodstuffs inside microwave ovens. By combining tablet and sample, an optimum heat distribution is achieved within the sample mixture inside the closed vessel. Surprisingly, this fine temperature control can be maintained during microwaving pulses for periods longer than 10 minutes. With the disclosed apparatus (10), method and tablet, DNA of higher analytical quality, i.e. free of PCR inhibitors, can be quickly and efficiently isolated from complex matrices. A simple, reliable and economically advantageous DNA extraction than conventional methods can thus be achieved.

In a preferred embodiment of the disclosure, the apparatus (10) and method for extracting nucleic acids may be used for isolating and characterizing the type of nucleic acids from raw and/or processed animal and plants materials and processed products thereof. Parameters for the detection of specific DNA by means of (real-time) PCR can be: 1) potential allergens e.g. cereals and products thereof, almond and products thereof, cashew and products thereof, peanut and products thereof, hazelnut and products thereof, macadamia and products thereof, mustard and products thereof, soya and products thereof, sesame and products thereof, walnut and products thereof, pistachio and products thereof, lupine and products thereof, celery and products thereof, fish and products thereof, crustaceans and products thereof; 2) genetically modified organisms GMO e.g. genetically modified maize, soy beans, rape seed, potatoes, tomatoes; 3) animal identification e.g. horse, pig, sheep, poultry; 4) plant identification e.g. apricot kernels, cherries, peaches; 5) pathogens e.g. Salmonella spp., Listeria spp. Shigella spp., Campylobacter spp., Cronobacter, Clostridium spp., Legionella spp., Enterobacteriaceae, Escherichia spp, fecal samples, 6) clinical investigations e.g. preferably human and veterinary samples like blood, feces, wounds, for the detection of pathogens like MRSA, viruses, bacteria; and 7) forensic samples such as swabs. Further embodiments, objects and advantages of the invention will become apparent from the following examples.

### EXAMPLES

### Example 1

### Apparatus and Composition for DNA Extraction

A closed cuboid chamber made of aluminum was built by conventional methods, having the following dimensions: length 45 cm; width 10 cm; and height 5 cm. One opening was drilled at the upper part of the chamber at one chamber's end to allow microwaves entering the chamber. A magnetron was used for generating microwaves. The magnetron was attached at the side with the opening so that microwaves could be generated and, immediately after, propagated through the opening into the chamber. The magnetron having a corresponding power supply was operated at a frequency of 2.45 GHz. A cooler (ventilator) was positioned closed to the magnetron for reducing the excessive temperature of the instrument upon operation. Four further openings separated by 9 cm each were drilled onto the upper part of the chamber. Next, a cylindrical metal piece of 2.5 cm with a diameter of 1 cm was soldered onto every opening. In order to tightly accommodate sample tubes at this spots, cylindrical holding pieces made of Teflon were produced so that they could fit inside the cylindrical piece and let a sample tube be tightly placed inside. The holding piece displayed an open upper end, whose edge extended sideways above the edge of the cylindrical metal piece. The bottom part was closed so that a sample tube could be held upright without falling into the chamber.

The microwavable samples tubes were made of plastic and could be closed by means of a screw cup. The tubes were introduced into the holding piece, so positioned that the edge of the bottom part of the sample tubes projected into the chamber approximately 1 mm with respect to the upper metal wall of the chamber. This layout guaranteed that only a fraction of the bottom part of the sample tube projecting into the chamber was directly exposed to the microwaves.

The sample tubes contained each 1 mL of distilled water and a DNA extraction tablet according to the following composition. Pharmaceutical grade talcum powder form was used in the preparation of the DNA extraction tablets. The talcum had a median particle size of 1.2 pm, a median diameter D50 of 0.65 pm and a density of 2.8 g/cm3. The talcum powder (hydrated magnesium silicate) had the following composition: SiO₂ (61.5%), MgO (31.0%), CaO (0.4%), Fe₂O₃ (0.6%), (Al₂O₃) 0.5%, with a pH of 8.8. The second component was phosphate buffered saline according to Dulbecco (1×PBS=NaCl 137 mmol/L, Na₂HPO₄⋅2 H₂O 10 mmol/L, KCl 2.7 mmol/l, KH₂PO₄ 2 mmol/L) and added as a microcrystalline salt. Pharmaceutical grade swellable microcrystalline cellulose free of contaminants was used as disintegration agent. All three components were compressed into a tablet using a stamping press. The “salt” tablet had a total unit weight of 117 mg and consisted of talcum particles: 20 mg (17.1%); crystalline PBS salt: 68 mg (58.1%); Swellable cellulose: 29 mg (24.8%). The tablet was sized for the extraction of food samples having about 200 mg.

In order to monitor the temperature inside every vessel at different time point, a measuring probe was introduced into each of the closed vessels from its upper part (through a thin opening on the lid) and sealed to avoid evaporation and spillage. After vortexing the mixture of water and tablet for 5 seconds, the samples tubes were placed on the apparatus as described above. Four different sample tubes at four positions were monitored. See FIG. 10; the magnetron was operated at pulses, whereby the first pulse was applied at power 270 W for 25 seconds. The temperature inside the closed tubes reached rapidly 100-105° C. without bursting. Further 5-10 second pulses at 130 W separated by 10-15 second rest intervals were applied to the samples, whereby the temperature was raised back to 90-105° C. without any sudden bursting of the tube content. This cycle was repeated 8 times. Importantly, when this experiment was performed with sample tubes containing water only, the sample tubes exploded virtually every time after the first pulse. This experiment demonstrated that the temperature of the vessel's content can only be rapidly increased in a controlled and safe manner by the synergistic activity of the apparatus and the DNA extraction “salt” tablet.

### Example 2

### Microwave-Mediated Processing of Food Samples

DNA extraction: 100 g of sample was obtained and mechanically homogenized using a grinder or mixer with rotating knifes. 200 mg homogenous sample was transferred into a 2 ml (microwave-transparent) plastic vial with screw cap or snap-lock using a spatula or pipette. A DNA extraction tablet according to Example 1 was added together with 1 ml aqua dest. After closing the vial, the tube was vortexed for 3 seconds and spinned down shortly to remove any liquid from the cap of the vessel. The vial containing the sample and the composition for DNA extraction was closed by screwing a screw cup onto the vessel and placed onto the stationary spots of the apparatus according to Example 1.

Next, the magnetron was operated and, thereby, the samples were irradiated with microwaves. None of the samples containing the “salt” tablet burst at any time period. All attempts to microwave samples without the tablet according to the disclosure resulted in explosive behaviors and sample spillage. For comparison, the samples were also processed using a conventional heat block. This approach required 20 minutes incubation periods at 99° C. for effective dissociation of the samples.

Following microwave irradiation or incubation in the heat block, the closed sample vessels were centrifuged at 14.000 rpm, RT for 5 minutes. After centrifugation, two well defined phases were observed in the dissociated sample preparations with added extraction tablet, displaying a clear supernatant and a defined precipitation pellet. The pellet with precipitated cell debris was discarded and the clear supernatant used for further analyses.

Desalting: The supernatant was desalted using a DNA affinity column (Centrispin, Genaxxon) according to the manufacturer's instructions. First, 100 μ20 supernatant was added to 500 μL binding buffer and vortexed. The volume (600 μL) loaded onto an equilibrated DNA spin column, followed by 14.000 rpm for 1 minute at RT. The flow-through was discarded; the column washed with 700 μL washing buffer and centrifuged 14.000 rpm for 1 minute at RT. The sample DNA was finally eluted with 50 μL elution buffer.

These experiments demonstrated that several samples contained in closed vials could be processed simultaneously at atmospheric pressure with a single apparatus, providing fine control of the temperature inside the vessel and, notably, in absence of vessel bursting and spillage.

### Example 3

### Conventional CTAB DNA Extraction

For comparative purposes, the homogenized samples were subjected to the standard CTAB genomic DNA extraction protocol. To this end, 100 ml CTAB lysis buffer was prepared by mixing 2.0 g CTAB (hexadecyl trimethylammonium bromide), 10.0 ml 1 M Tris pH 8.0, 4.0 ml 0.5 M EDTA pH 8.0, 28.0 ml 5 M NaCl, 40.0 ml H₂O. The pH was adjusted with HCl to pH 8.0 and aqua dest. added up to a volume of 100 ml. 2 g homogenized sample was mixed with 10 ml CTAB lysis buffer and 25 μL proteinase K (20 mg/ml) and incubated overnight at 60 degrees Celsius under mild shaking. Following centrifugation at 4000 g, RT for 5 minutes, the first pellet was discarded and the supernatant again centrifuged at 14000 g, RT for 10 minutes. The supernatant was then extracted with an equal volume of chloroform. 600 pi aqueous phase was mixed with 1.2 ml CTAB precipitation buffer (5 g/L CTAB, 0,04 mol/L NaCl), the DNA precipitated at room temperature for 60 minutes, followed by centrifugation at 14000 g for 10 minutes at room temperature. The supernatant was discarded and the DNA pellet taken up in 350 pi 1.2 mol/l NaCl solution. After another extraction with 350 pi chloroform, the DNA in the aqueous phase was again isopropanol precipitated at room temperature for 20 minutes. After centrifugation, the supernatant was discarded and the DNA pellet spin-washed with 500 pi cold 70% ethanol and dried at ambient temperature. The dried DNA pellet was dissolved in 100 μL 0.1× TE. RT-PCR and Ct value determination was performed as described in Example 4.

### Example 4

### Real-Time PCR and Assessment of DNA Extraction

Real-time PCR was performed using the RotorGene thermocycler (Qiagen) in accordance with manufacturer's instructions. The PCR was performed in a 20 μL volume comprising 10 μL 2× SensiFAST™ Multiplex Master Mix (Bioline GmbH, Luckenwalde, DE), 10 μL DNA extract, 400 nM primers, and 200 nM reference DNA. The SensiFast Multiplex MasterMix consists of a buffer system, dNTPs, Mg₂+, and DNA polymerase. The PCR thermocycler program consisted of an incubation step at 95° C. for 5 min followed by 45 cycles of incubation at 95° C. for 15 sec, 60° C. for 15 sec and 72° C. for 10 sec. PCR were performed in duplicates. The Ct value was determined using a threshold of 0.02 by means of the RotorGene software.

For assessment of DNA extraction, 2 ng DNA extracted and prepared in accordance with Examples 2 and 3 was added into a volume of 10 μL with specific primers for detection of target DNA. The relative PCR sensitivity can then be taken from the Ct values.

### Example 5

### Detection of Foreign Plant DNA in Plant Material

Parsley samples containing known amounts of celery (1000 ppm) were homogenized, DNA extracted and isolated according to Examples 2 and 3, and analysed according to Example 4. Primers for detection of celery were used.

Table 1 shows Ct values obtained with samples processed a) with the apparatus and tablet (“Salt”) of the disclosure at 200 W power for 3 different time periods (10 s, 20 s and 2 min); b) with a heat block at 99° C. for 20 minutes and the tablet (“Salt”) of the disclosure; or c) following the CTAB protocol (“CTAB”). When using the apparatus of the disclosure, experiments were performed placing a sample tube on each of the four stationary spots of the chamber for every time period. Shown are average values.

The result of the extraction experiments showed in Table 1 exhibited almost identical Ct values regardless of the irradiation times, namely for 10 or 20 seconds. Longer exposure (6-12 times) to microwaves resulted in similar Ct values. This indicates that the disclosed apparatus and tablet allows for reproducible purification of high quality DNA in an unprecedented short period of time. The nucleic acid extraction with the disclosed apparatus may only be carried in combination with the disclosed tablet for DNA extraction. With the disclosed method, homogenous heating of samples for DNA extraction by means of microwave radiation can thus be achieved, without the disadvantage of vial bursting and spillage. In addition, many samples can be processed very rapidly and, notably, without the risk of cross-contamination due to sudden opening of lids and/or vial explosion. Importantly, the Ct values were comparable to those obtained when DNA was extracted with the standard CTAB protocol. The results in Table 1 demonstrate that an efficient analysis of samples can be performed within a fraction of the time required by conventional methods. Further comparative analysis, consisting in the application of heat with a thermoblock instead of microwaving, corroborated that the fast transfer of heat with the disclosed apparatus improved the performance of the PCR reaction.

The disclosed method renders DNA extraction a less laborious procedure with the further advantage that it does not require the use of expensive and/or hazardous chemicals. No chaotropic chemicals or organic solvents are required, so that the requirement of a laboratory fume hood is dispensed. The use of the disclosed extraction composition also contributes to the reduction of the risks of carrying over potential exogenous PCR polymerase inhibitors. Although not essential, the salt contained in the supernatant following extraction was effectively removed by DNA affinity columns prior PCR reaction.

TABLE 1

| | FOREIGN | HEAT | | | Ct | Ct |
|---|---|---|---|---|---|---|
| SAMPLE | MATERIAL | TRANSFER | TIME | | Salt | CTAB |
| Parsley | Celery 1000 ppm | 200 W | 10 | s | 28.99 | 29.78 |
| Parsley | Celery 1000 ppm | 200 W | 20 | s | 28.58 | 29.47 |
| Parsley | Celery 1000 ppm | 200 W | 2 | min | 30.27 | 29.28 |
| Parsley | Celery 1000 ppm | Heat block | 20 | min | 29.57 | 28.93 |
| | | 99° C. | | | | |
| Parsley | Celery 1000 ppm | Heat block | 20 | min | 30.71 | 29.46 |
| | | 99° C. | | | | |
| Parsley | Celery 1000 ppm | Heat block | 20 | min | 30.37 | 29.48 |
| | | 99° C. | | | | |

### Example 6

### Detection of Genetically Modified Organisms

Cornmeal containing foreign genetic material ((CaMV-35S-promoter, originated from cauliflower mosaic virus) was homogenized, DNA extracted and isolated according to Example 2, and analysed according to Example 4. The target DNA was tested for contamination with genetically modified Roundup Ready™ soya by the presence of 35S promoter. Pairs of suitable primers for detection of 35S were used.

Table 2 shows Ct values obtained with samples processed a) with the apparatus and tablet (“Salt) of the disclosure at 200 W for 3 different time periods (10 s, 20 s and 2 min). For comparison, homogenized samples were incubated on a heat block at 99 ° C. for 20 minutes with the tablet of the disclosure. When using the apparatus of the disclosure, experiments were performed placing a sample tube on each of the four stationary spots of the chamber for every time period. Shown are average values.

These experiments demonstrate a reproducible detection sensibility of transgenic DNA extracted with the apparatus and tablet of the disclosure upon exposure to microwaves for several seconds. Further exposure to microwaves (2 min) did not result in adverse effects but nearly identical values upon PCR analysis. Similar Ct values were obtained by incubation of the sample in a heating block, however, for 20 minutes. The results in Table 2, which exhibit extremely low Ct values obtained by the method of the disclosure, highlight the quality of samples extracted with the disclosed apparatus and tablet, particularly regarding the virtual absence of DNA polymerase inhibitors and the extremely fast extraction procedure.

TABLE 2

| MATRIX | TRANSGENE | HEAT TRANSFER | TIME | | Ct Salt |
|---|---|---|---|---|---|
| Cornmeal | 35S promoter | 200 W | 10 | s | 20.47 |
| Cornmeal | 35S promoter | 200 W | 20 | s | 19.04 |
| Cornmeal | 35S promoter | 200 W | 2 | min | 19.29 |
| Cornmeal | 35S promoter | Heat block 99° C. | 20 | min | 19.41 |

### Example 7

### Detection of Genetically Modified Organisms in Complex Matrices

Animal feed, wheat flour and cornmeal were homogenised, DNA extracted and isolated according to Examples 2 and 3, and analysed according to Example 4 for the presence of foreign genetic material. Pairs of suitable primers for 15 detection of 35S promoter, Roundup Ready™ soya, and NOS promoter (from Agrobacterium) were used.

Table 3 shows Ct values obtained with samples processed a) with the apparatus and tablet (“Salt”) of the disclosure at 200 W for 15 seconds; b) with a heat block at 99° C. for 20 minutes and the tablet (“Salt”) of the disclosure; or c) following the 20 CTAB protocol (“CTAB”). When using the apparatus of the disclosure, experiments were performed placing a sample tube on each of the four stationary spots of the chamber for every time period. Shown are average values.

The results in Table 3 confirm the feasibility of efficient DNA extraction and detection from different matrices by the method and apparatus of the present disclosure. Notably, a simultaneous analysis of multiple (trans)genes is possible, regardless of the matrix from which the DNA was extracted. Importantly, the sensibility of DNA detection resulting from the extraction procedure of the present disclosure is comparable, and even better, to the standard method CTAB. Advantageous are also the extremely short time periods for extraction of transgenic material compared to the use of heat blocks or the CTAB method.

TABLE 3

| | TRANSGENIC | Ct Salt | Ct Salt | Ct |
|---|---|---|---|---|
| MATRIX | MATERIAL | Microwave | Heat block | CTAB |
| Animal feed | 35S promoter | 20.17 | 20.66 | 23.12 |
| Wheat flour | Roundup Ready ™ | 36.52 | 35.93 | 36.87 |
| Cornmeal | NOS promoter | 30.73 | 30.81 | 30.07 |

### Example 8

### Detection of Animal Material in Complex Matrices

Pizza salami, minced meat and meat ball were homogenised, DNA extracted and isolated according to Examples 2 and 3, and analysed according to Example 4 for the presence of animal material. Pairs of suitable primers for detection of beef and pork were used.

Table 4 shows Ct values obtained with samples processed a) with the apparatus and tablet (“Salt”) of the disclosure at 200 W for 15 seconds; b) with a heat block at 99° C. for 20 minutes and the tablet (“Salt”) of the disclosure; or c) following the CTAB protocol (“CTAB”). When using the apparatus of the disclosure, experiments were performed placing a sample tube on each of the four stationary spots of the chamber for every time period. Shown are average values.

TABLE 4

| | | Ct Salt | Ct Salt | Ct |
|---|---|---|---|---|
| MATRIX | ANIMAL | Microwave | Heat block | CTAB |
| Pizza salami | Beef | 26.72 | 29.51 | 29.82 |
| Minced meat | Beef | 25.97 | 25.42 | 26.57 |
| Meat ball | Beef | 24.76 | 24.57 | 27.09 |
| Meat ball | Pork | >45 | >45 | >45 |

The results in Table 4 show the advantageous animal DNA extraction and detection from complex matrices, such as mixtures of carbohydrates, fats and proteins (pizza salami), by the method and apparatus of the present disclosure with comparable efficiency regardless of the matrix. A simultaneous analysis of different animal materials was performed, regardless of the matrix from which the DNA was extracted. A remarkable improved sensibility of animal DNA detection resulted from the extraction procedure of the present disclosure in comparison to the standard method CTAB. Advantageous are also the short time periods for extraction of animal material with the aid of microwaves compared to the use of heat blocks or the CTAB method

### Example 9

### Detection of Allergens in Complex Matrices

Chocolate, celery seed powder, millet, flavoring, couscous and coriander were homogenized, and DNA extracted and isolated according to Examples 2 and 3, and analysed according to Example 4 for the presence of allergens. Pairs of suitable primers for detection of almond, peanut, celery, soya and walnut were used.

Table 5 shows Ct values obtained with samples processed a) with the apparatus and tablet (“Salt”) of the disclosure at 200 W for 15 seconds; b) with a heat block at 99° C. for 20 minutes and the tablet (“Salt”) of the disclosure; or c) following the CTAB protocol (“CTAB”). When using the apparatus of the disclosure, experiments were performed placing a sample tube on each of the four stationary spots of the chamber for every time period. Shown are average values.

The results in Table 5 confirm the feasibility of the fast and efficient detection of allergens from complex matrices of different nature by the method and apparatus of the present disclosure. A simultaneous analysis of multiple allergens is thus possible, regardless of the matrix from which the DNA was extracted. Notably, the sensibility of detection resulting from the extraction procedure of the present disclosure is improved in virtually all tested matrices, in comparison to the standard method CTAB. Advantageous are also the extremely short time periods for extraction of allergens compared to the use of heat blocks or the CTAB method.

TABLE 5

| | | Ct Salt | Ct Salt | Ct |
|---|---|---|---|---|
| MATRIX | ALLERGEN | Microwave | Heat block | CTAB |
| Chocolate | Almond | 30.06 | 32.22 | 32.81 |
| Chocolate | Peanut | 24.14 | 24.57 | 25.13 |
| Celery seed powder | Celery | 15.71 | 15.42 | 16.24 |
| Millet | Soya | 32.02 | 31.24 | 31.44 |
| Flavouring | Celery | 29.98 | 31.84 | 29.83 |
| Couscous | Celery | 32.07 | 33.23 | 34.27 |
| Coriander | Walnut | 20.42 | 22.77 | 21.52 |

### Example 10

### Influence of the Composition for DNA Extraction

Hazelnut, walnut and soya samples containing known amounts of chocolate (10000 ppm) were homogenized, and DNA extracted and isolated according to Example 2, and analysed according to Example 4.

Table 6 shows Ct values obtained with samples processed with the apparatus and tablet (“Salt”) of the disclosure at 125 W and 150 W for 2 different time periods (30 s and 60 s). For comparison, homogenized samples were a) processed with the disclosed apparatus at 150 W for 60 seconds, with no tablet, but crystalline PBS salt 68 mg/mL; and b) incubated on a heat block at 99° C. for 20 minutes with the tablet of the disclosure. Experiments were performed placing the samples on two of the four stationary spots (positions P3 and P4, respectively) of the chamber for each condition.

Table 6 shows that comparable Ct values are obtained regardless of the position of the samples in the apparatus, especially at 125 W and 60 seconds. It is further shown that there is a correlation between power and time, whereby lower power values require longer time periods for DNA extraction. However, Ct values are generally better when the apparatus is operated at lower power. This demonstrates that the disclosed apparatus requires low energy consumption for achieving efficient DNA extraction.

TABLE 6

| | | | | |
|---|---|---|---|---|
| Extraction | Tablet | Tablet | PBS only | Tablet |

Hazelnut (Chocolate 10000 ppm)

| | | | | | | | |
|---|---|---|---|---|---|---|---|
| Heat transfer | 150 | W | 125 | W | 150 | W | Heat block 99° C. |
| Time | 30 | sec | 60 | sec | 60 | sec | 20 Min |

| | | | | |
|---|---|---|---|---|
| Ct (P3) | 29.15 | 27.21 | 31.93 | 27.25 |
| Ct (P4) | 30.06 | 27.03 | 31.85 | 27.54 |

Walnut (Chocolate 10000 ppm)

| | | | | | | | |
|---|---|---|---|---|---|---|---|
| Heat transfer | 150 | W | 125 | W | 150 | W | Heat block 99° C. |
| Time | 30 | sec | 60 | sec | 60 | sec | 20 Min |

| | | | | |
|---|---|---|---|---|
| Ct (P3) | 29.15 | 27.21 | 31.93 | 27.25 |
| Ct (P4) | 30.06 | 27.03 | 31.85 | 27.54 |

Soya (Chocolate 10000 ppm)

| | | | | | | | |
|---|---|---|---|---|---|---|---|
| Heat transfer | 150 | W | 125 | W | 150 | W | Heat block 99° C. |
| Time | 30 | sec | 60 | sec | 60 | sec | 20 Min |

| | | | | |
|---|---|---|---|---|
| Ct (P3) | 27.59 | 25.25 | 30.10 | 25.58 |
| Ct (P4) | 27.55 | 25.30 | 31.18 | 25.55 |

The presence of magnesium silicate is according to Table 6 essential for an efficient DNA extraction and PCR polymerase reaction. Only at very low power, i.e. 150 W or lower, the samples tubes remain intact when magnesium silicate is not present in the sample mixture. Attempts to operate the apparatus at a power higher than 150 W without magnesium silicate in the sample mixture resulted in bursting of the sample tubes. Furthermore, the efficiency of extraction is extremely low in comparison to sample mixtures containing magnesium silicate. In every experiment, the added magnesium silicate had adsorbed and precipitated DNA polymerase inhibitors present in the matrices and, thus, the PCR polymerase reaction was carried out efficiently. The addition of magnesium silicate further facilitates the handling of samples containing plenty of phospholipids, fatty acids and triglycerides.

1. Apparatus for converting electromagnetic microwave radiation into thermal energy, comprising
a solid state microwave generator, which generates and emits microwaves using power semiconductor devices;
a chamber, which is connected to the generator and confines the emitted microwaves and wherein the microwaves adopt a monomode pattern;
a plurality of stationary spots, which are fixedly attached onto the upper part of the chamber and project inwards, wherein the stationary spots are longitudinally separated from each other by identical intervals of predetermined distance, and the emitted microwave radiation propagates within the chamber as a standing wave, and wherein the said predetermined distance is half the wavelength of the emitted microwave radiation.

2. (canceled)

3. Apparatus according to claim 1, wherein each spot comprises on outer wall made of microwave-reflecting material and an inner holding piece made of a microwave transparent material selected from plastic, silicon carbide, resin, Teflon®, polytetrafluoroethylene, perfluoroalkoxy, fluorinated ethylene propylene, ceramics and combinations thereof.

4. Apparatus according to claim 1, wherein the spots project inwards into the chamber from about 0.01 mm to about 10 mm, preferably from about 0.1 mm to about 5 mm, preferably from about 0.5 mm to about 2 mm, to limit the direct exposure to microwaves to the lower portion of the sample tube protruding into the chamber.

5. Apparatus according to claim 1, wherein the chamber displays cuboid shape with a length from about 40 to about 60 cm, a width from about 8 cm to about 12 cm, and a height from about 4 cm to about 8 cm.

6. Apparatus according to claim 1, wherein a plurality of chambers with a plurality of stationary spots located longitudinally are connected to each other and the electromagnetic microwave radiation propagates into the plurality of chambers through a plurality of slits or openings on the walls of said chambers.

7. Apparatus according to claim 1, wherein the chamber displays circular shape and said stationary spots are located circularly along the chamber.

8. Apparatus according to claim 1, wherein the chamber is made of microwave-reflecting material selected from metal, aluminium, stainless steel, or combinations thereof.

9. Method of isolating nucleic acids for subsequent analysis by a DNA polymerase chain reaction encompassing the conversion of microwave radiation into thermal energy, comprising the steps of:
providing an apparatus for converting microwave radiation into thermal energy, comprising:
a solid state microwave generator, which generates and emits microwaves using power semiconductor devices;
a chamber, which is connected to the generator and confines the emitted microwaves and wherein the microwaves adopt a monomode pattern; and
a plurality of stationary spots, which are fixedly attached onto the upper part of the chamber and project inwards, wherein the stationary spots are longitudinally separated from each other by identical intervals of predetermined distance, and the emitted microwave radiation propagates within the chamber as a standing wave, and wherein the said predetermined distance is half the wavelength of the emitted microwave radiation;
providing a plurality of sample tubes with closable means, which are detachably connected to the plurality of stationary spots; and loading each sample tube with (i) a sample to be analysed, (ii) a tablet comprising water insoluble hydrated magnesium silicate and crystalline phosphate buffer saline, and (iii) water to obtain a weight ratio of tablet to sample in the vessel from 1:5 to 5:1; and closing the vessel;
placing each closed sample tube into each stationary/immobile spots; operating the microwave generator so that within the chamber an standing microwave is generated contacting the plurality of stationary/immobile spots;
propagating microwave radiation through the plurality of stationary/immobile spots into the plurality of sample tubes, whereby thermal energy is generated, so that a temperature rise of the mixture inside the closed vessel in the range from 85° C. to 140° C. is elicited and nucleic acids are released; and
optionally separating the water-insoluble components adsorbed on the magnesium silicate and removal of the supernatant containing soluble nucleic acids, followed by desalting to obtain a solution of nucleic acids suitable for PCR analysis.

10. Method of claim 9, further comprising the steps of dissolving a tablet or capsule of solid buffer components to obtain an aqueous phosphate buffered saline solution having a pH from 5.5 to 7.0 and a salt concentration of 0.4 to 1.2 mol/L; and operating the microwave generator for about 5 seconds to 2 minutes.

11. Method of claim 10, wherein the tablet or capsule comprises a non-aqueous mixture of solids comprising from 30 to 70 percent by weight crystalline phosphate buffer saline; and from 10 to 40 percent by weight water insoluble hydrated magnesium silicate particles.

12. Method of claim 9, wherein the solid tablet or capsule for extracting nucleic acids further comprises from 15 to 45 percent by weight hydrophilic colloid, wherein the hydrophilic colloid is cellulose, carboxy-methyl cellulose, cellulose derivatives, alginate, starch, xantan gum, arabic gum, guar gum or mixtures thereof.

13. Use of the method of claim 9 for isolating and characterizing nucleic acids from any one of raw and/or processed animal and plants materials, processed products thereof, human and veterinary samples and forensic samples.
